(19) United States
(12) Patent Application Publication
(10) Pub. No.: US 2026/0085272 A1
(43) Pub. Date: Mar. 26, 2026

(54) MICROORGANISM POSITIVE BOTTLE PROCESSING APPARATUS

(71) Applicant: bioMérieux Shanghai Company LTD, Shanghai (CN)

(72) Inventors: Jiafeng LUO, Shanghai (CN); Xiaokun DONG, Shanghai (CN); Wei ZHANG, Shanghai (CN)

(73) Assignee: bioMérieux Shanghai Company LTD, Shanghai (CN)

(21) Appl. No.: 19/340,211

(22) Filed: Sep. 25, 2025

(30) Foreign Application Priority Data
Sep. 25, 2024 (CN) 2024113370157

(51) Int. Cl. C12M 1/36; C12M 1/00; C12M 1/12; C12M 1/22; G01N 35/00

(52) U.S. Cl. CPC C12M 41/48; C12M 23/10; C12M 37/00; C12M 41/14; G01N 35/00069; G01N 35/0099; G01N 2035/00138

(57) ABSTRACT

A microorganism positive bottle processing apparatus includes an input vertical mechanism, sterilization mechanism, scanning mechanism, glass slide placement mechanism, translation and turning mechanism, plate placement mechanism, transferring robotic arm, antimicrobial susceptibility disc dispenser, needle supply mechanism, plate incubation mechanism and plate loading apparatus. The sterilization mechanism is spaced from the input vertical mechanism; the scanning mechanism is spaced from the input vertical mechanism; the translation and turning mechanism is on the scanning mechanism remote side; the plate placement mechanism is on one side of the translation and turning mechanism, and the glass slide placement mechanism is spaced from the plate placement mechanism lengthwise from the translation and turning mechanism; the transferring robotic arm is on one side of the input vertical mechanism; the transferring robotic arm moves a positive bottle from the input vertical mechanism to the scanning mechanism, then to the translation and turning mechanism, and for puncturing.

### TECHNICAL FIELD

The present invention relates to the technical field of organism sample processing, in particular a microorganism positive bottle processing apparatus.

### BACKGROUND ART

Existing microorganism positive bottle processing is mainly manual, which requires a lot of labour power and different devices to perform preprocessing, wasting labour power and material resources.

In summary, how to design a processing apparatus for a microorganism positive bottle, to reduce the workload of people, is a problem to be urgently solved for those skilled in the art at present.

### SUMMARY OF THE INVENTION

The main object of embodiments of the present invention is to propose a microorganism positive bottle processing apparatus, with the intention of designing an apparatus that automatically processes a positive bottle.

The technical solution of the present invention that solves the above technical problem is to provide a microorganism positive bottle processing apparatus, comprising: an input vertical mechanism, a sterilization mechanism, a scanning mechanism, a translation and turning mechanism, a plate placement mechanism, a plate supply mechanism, a plate lid-opening mechanism, a glass slide placement mechanism, a transferring robotic arm, an antimicrobial susceptibility disc dispenser, a needle supply mechanism, a plate incubation mechanism and a plate loading apparatus; the sterilization mechanism is spaced apart from the input vertical mechanism, and the sterilization mechanism faces a vertical positioning recessed block of the input vertical mechanism; the scanning mechanism is spaced apart from the input vertical mechanism; the translation and turning mechanism is arranged on one side of the scanning mechanism that is remote from the input vertical mechanism; the plate placement mechanism is arranged on one side of the translation and turning mechanism in a length direction of the translation and turning mechanism, and the glass slide placement mechanism is spaced apart from the plate placement mechanism in the length direction of the translation and turning mechanism; the transferring robotic arm is arranged on one side of the input vertical mechanism; the transferring robotic arm is used for moving a positive bottle from the input vertical mechanism to the scanning mechanism, moving the positive bottle from the scanning mechanism to the translation and turning mechanism, and for puncturing the positive bottle.

Furthermore, the input vertical mechanism comprises: a sliding tube, a vertical lifting mechanism, a detection photoelectric pair and a vertical positioning recessed block, the sliding tube being an arc-shaped mechanism, used for a positive bottle to slide, the vertical lifting mechanism being arranged at one end of the sliding tube, and a piston of a pneumatic cylinder of the vertical lifting mechanism facing the sliding tube, the detection photoelectric pair being arranged on the vertical lifting mechanism, and being located on one side of the sliding tube, and the detection photoelectric pair being electrically connected to the pneumatic cylinder of the vertical lifting mechanism; and the vertical positioning recessed block being arranged on one side of the vertical lifting mechanism that is remote from the sliding tube.

Furthermore, the microorganism positive bottle processing apparatus further comprises two rotation mechanisms, the two rotation mechanisms being respectively arranged below the vertical positioning recessed block, used for driving rotation of a positive bottle inside the vertical positioning recessed block, and below the scanning mechanism, used for driving rotation of the positive bottle on the scanning mechanism; the rotation mechanism comprises: a rotating pulley, a stepper motor and a rotation photoelectric pair, the rotating pulley being arranged below the vertical positioning recessed block and below the scanning mechanism, the stepper motor being spaced apart from the rotating pulley, and being connected thereto by means of a drive belt, and the rotation photoelectric pair being arranged on one side of the rotating pulley, and the rotation photoelectric pair being electrically connected to the stepper motor.

Furthermore, the translation and rotation mechanism comprises a sliding rail, an electric rotating platform, a rotating electric motor, a pneumatic cylinder and a liquid-dripping rotating gripper, the sliding rail being arranged on one side of the scanning mechanism, the electric rotating platform being movably arranged on the sliding rail, the rotating electric motor being rotatably arranged on the electric rotating platform, the pneumatic cylinder being arranged on the rotating electric motor, and the liquid-dripping rotating gripper being arranged on a piston of the pneumatic cylinder.

Furthermore, the microorganism positive bottle processing apparatus further comprises a plate lid-opening mechanism and a plate supply mechanism, the plate lid-opening mechanism being arranged on one side of the plate placement mechanism, and the plate supply mechanism being spaced apart from the plate placement mechanism.

Furthermore, the microorganism positive bottle processing apparatus further comprises: a workbench and a needle supply mechanism, wherein the input vertical mechanism, the sterilization mechanism, the scanning mechanism, the translation and turning mechanism, the plate placement mechanism, the plate lid-opening mechanism, the plate supply mechanism, the glass slide placement mechanism and the rotating robotic arm are all arranged on the workbench, and the needle supply mechanism is arranged on the workbench.

Furthermore, the microorganism positive bottle processing apparatus further comprises: an antimicrobial susceptibility disc dispenser, a plate incubation mechanism and a plate loading apparatus, the antimicrobial susceptibility disc dispenser being arranged on the workbench, the plate loading apparatus being arranged on a lower side of the workbench, used for supplying a plate to the plate supply mechanism, and the plate incubation mechanism being spaced apart from the workbench, wherein the plate incubation mechanism includes a constant-temperature incubator and a carbon dioxide incubator, the temperature of both being 35±2° C.

In the technical solution of the present invention, a bottle is directly fed into the input vertical mechanism, and is vertically processed; the vertically oriented positive bottle is sterilized by the sterilization mechanism; the sterilized positive bottle is moved by means of the transferring robotic arm to the scanning mechanism for scanning processing; the scanned positive bottle is moved by means of the transferring robotic arm to a liquid-dripping rotating gripper of the translation and turning mechanism; the transferring robotic arm punctures the positive bottle on the liquid-dripping rotating gripper; the punctured positive bottle is moved by means of the translation and turning mechanism to an upper side of the plate placement mechanism; rotation of the translation and turning mechanism causes the positive bottle to drip liquid on a plate on the plate placement mechanism; the translation and turning mechanism is rotated until the positive bottle is vertical, and the translation and turning mechanism moves the positive bottle; the transferring robotic arm draws a line on the plate, presses an antimicrobial susceptibility disc in the antimicrobial susceptibility disc dispenser onto the MH plate, and transfers all processed plates to a culture in the incubation mechanism; the positive bottle is moved to an upper side of the glass slide placement mechanism; rotation of the translation and turning mechanism causes the positive bottle to drip liquid on a glass slide on the glass slide placement mechanism; the translation and turning mechanism is rotated until the positive bottle is vertical; the translation and turning mechanism moves the positive bottle; and the transferring robotic arm draws a line on the glass slide, to complete processing of the positive bottle, realizing automatic processing of the positive bottle.

### BRIEF DESCRIPTION OF THE DRAWINGS

To more clearly explain the technical solutions in the prior art or embodiments of the present invention, accompanying drawings that are needed in the description of the prior art or embodiments are outlined below. Obviously, the accompanying drawings in the description below are merely some embodiments of the present invention; for a person skilled in the art, according to the mechanisms shown in these drawings, other drawings could be further obtained without expending inventive effort.

FIG. 1 is a three-dimensional mechanism schematic drawing of the microorganism positive bottle processing apparatus of the present invention;

FIG. 2 is a front mechanism schematic drawing of the microorganism positive bottle processing apparatus of the present invention;

FIG. 3 is mechanism schematic drawing of the transferring robotic arm of the present invention;

FIG. 4 is a mechanism schematic drawing of the input vertical mechanism of the present invention;

FIG. 5 is a mechanism schematic drawing of the rotation mechanism of the present invention;

FIG. 6 is a mechanism schematic drawing of the translation and turning mechanism of the present invention;

FIG. 7 is a mechanism schematic drawing of the plate incubation mechanism of the present invention;

FIG. 8 is a mechanism schematic drawing of the plate output mechanism of the present invention;

FIG. 9 is a structural schematic drawing of the plate loading apparatus of the present invention.

Key to the figures:

| Number | Name |
|---|---|
| 10 | Input vertical mechanism |
| 11 | Sliding tube |
| 12 | Vertical lifting |
| | mechanism |
| 13 | Detection photoelectric |
| | pair |
| 14 | Vertical positioning |
| | recessed block |
| 20 | Sterilization mechanism |
| 30 | Scanning mechanism |
| 40 | Translation and turning |
| | mechanism |
| 41 | Sliding rail |
| 42 | Electric rotating |
| | platform |
| 43 | Rotating electric motor |
| 44 | Pneumatic cylinder |
| 45 | Liquid-dripping rotating |
| | gripper |
| 50 | Plate placement |
| | mechanism |
| 60 | Glass slide placement |
| | mechanism |
| 70 | Transferring robotic arm |
| 80 | Rotation mechanism |
| 81 | Rotating pulley |
| 82 | Stepper motor |
| 83 | Drive belt |
| 84 | Rotation photoelectric |
| | pair |
| 91 | Workbench |
| 92 | Needle supply mechanism |
| 93 | Plate lid-opening |
| | mechanism |
| 94 | Plate supply mechanism |
| 95 | Antimicrobial |
| | susceptibility disc |
| | dispenser |
| 96 | Plate incubation |
| | mechanism |
| 97 | Plate output mechanism |
| 98 | Plate loading assembly |
| 981 | Plate storage assembly |
| 982 | Plate translation |
| | assembly |
| 983 | Plate extraction |
| | assembly |
| 984 | Raising assembly |

### PARTICULAR EMBODIMENTS

The technical solutions in the embodiments of the present invention will be described clearly and fully below in view of the accompanying drawings in the Drawings of the description of the present invention. Obviously, the described embodiments are only some, not all, embodiments of the present invention. On the basis of the embodiments in the present invention, all other embodiments obtained by a person skilled in the art without inventive effort shall fall within the scope of protection of the present invention.

It must be explained that all directional indications (such as upper, lower, left, right, front, back, etc.) in the embodiments of the present invention are only for explaining relative positional relationships, movement situations, etc. of various components in a certain special state (such as shown in a figure); if this special state changes, then the directional indication also changes accordingly.

In addition, descriptions such as relating to “first”, “second”, etc. in the present invention are only used for descriptive purposes, and should not be construed as indicating or implying relative importance, or implicitly indicating a number of the technical features referred to. Thus, features defined with “first” or “second” may clearly or implicitly comprise at least one of this feature. In the description of the present invention, “several” and “multiple” mean at least two, such as two, three, etc., unless another specific definition is made explicit.

In the present invention, unless otherwise explicitly specified and defined, terms such as “connect”, “fix”, etc. should be understood in a broad sense. For example, “connect” may refer to: a fixed connection, a detachable connection or forming an integral whole; a mechanical connection or an electrical connection; a direct connection or an indirect connection via an intermediate medium; and communication between the interiors of two elements or an interactive relationship between two elements, unless otherwise explicitly defined. For a person skilled in the art, the specific meanings of such terms in the present invention may be understood according to the specific situation.

In addition, the technical solutions of the embodiments of the present invention may be combined with each other, but the combination must be implementable by a person skilled in the art; if a combination of technical solutions gives rise to a contradiction or cannot be implemented, then such a combination of technical solutions should be deemed not to exist, and is also not within the scope of protection of the present invention.

The present invention proposes a microorganism positive bottle processing apparatus, with the intention of designing an apparatus that automatically processes a positive bottle.

Below, the microorganism positive bottle processing apparatus proposed by the present invention will be illustrated in particular embodiments:

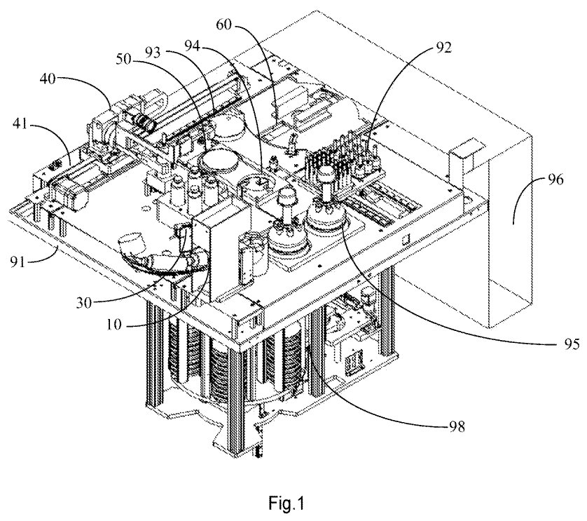

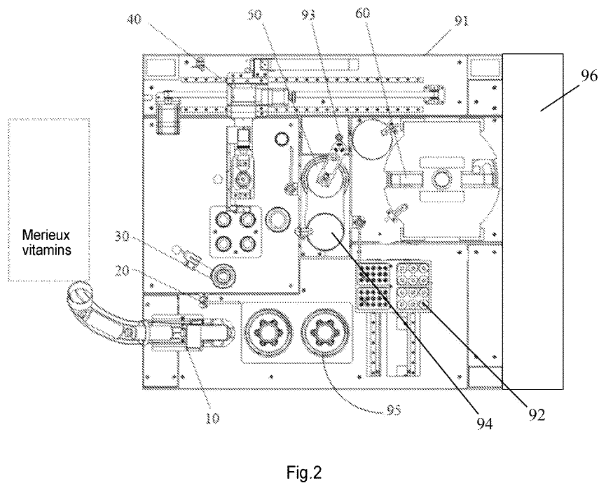

In a technical solution of the present embodiment, as shown in FIGS. 1 and 2, a microorganism positive bottle processing apparatus comprises: an input vertical mechanism 10, a sterilization mechanism 20, a scanning mechanism 30, a glass slide placement mechanism 60, a translation and turning mechanism 40, a plate placement mechanism 50 and a transferring robotic arm 70; the scanning mechanism 20 is spaced apart from the input vertical mechanism 10, and the sterilization mechanism 20 faces a vertical positioning recessed block 14 of the input vertical mechanism 10; the scanning mechanism 30 is spaced apart from the input vertical mechanism 10; the translation and turning mechanism 40 is arranged on one side of the scanning mechanism 30 that is remote from the input vertical mechanism 10; the plate placement mechanism 50 is arranged on one side of the translation and turning mechanism 40 in a length direction of the translation and turning mechanism 40, and the glass slide placement mechanism 60 is spaced apart from the plate placement mechanism 50 in the length direction of the translation and turning mechanism 40; the transferring robotic arm 70 is arranged on one side of the input vertical mechanism 10, and the transferring robotic arm 70 is used for moving a positive bottle from the input vertical mechanism 10 to the scanning mechanism 30, moving the positive bottle from the scanning mechanism 30 to the translation and turning mechanism 40, and for puncturing the positive bottle.

It will be understood that after a Merieux microorganism machine ejects a positive bottle, the positive bottle is directly fed into the input vertical mechanism 10, and is vertically processed; the vertically oriented positive bottle is sterilized by the sterilization mechanism 20; the sterilized positive bottle is moved by means of the transferring robotic arm 70 to the scanning mechanism 30 for scanning processing; the scanned positive bottle is moved by means of the transferring robotic arm 70 to a liquid-dripping rotating gripper 45 of the translation and turning mechanism 40; the transferring robotic arm 70 punctures the positive bottle on the liquid-dripping rotating gripper 45; the punctured positive bottle is moved by means of the translation and turning mechanism 40 to an upper side of the plate placement mechanism 50; rotation of the translation and turning mechanism 40 causes the positive bottle to drip liquid on a plate on the plate placement mechanism 50; the translation and turning mechanism 40 is rotated until the positive bottle is vertical, and the translation and turning mechanism 40 moves the positive bottle; the transferring robotic arm 70 draws a line on the plate, presses an antimicrobial susceptibility disc in an antimicrobial susceptibility disc dispenser 95 onto the MH plate, and transfers all processed plates to a culture in a plate incubation mechanism 96; the positive bottle is moved to an upper side of the glass slide placement mechanism 60; rotation of the translation and turning mechanism 40 causes the positive bottle to drip liquid on a glass slide on the glass slide placement mechanism 60; the translation and turning mechanism 40 is rotated until the positive bottle is vertical; the translation and turning mechanism 40 moves the positive bottle; and the transferring robotic arm 70 draws a line on the glass slide, to complete processing of the positive bottle, realizing automatic processing of the positive bottle.

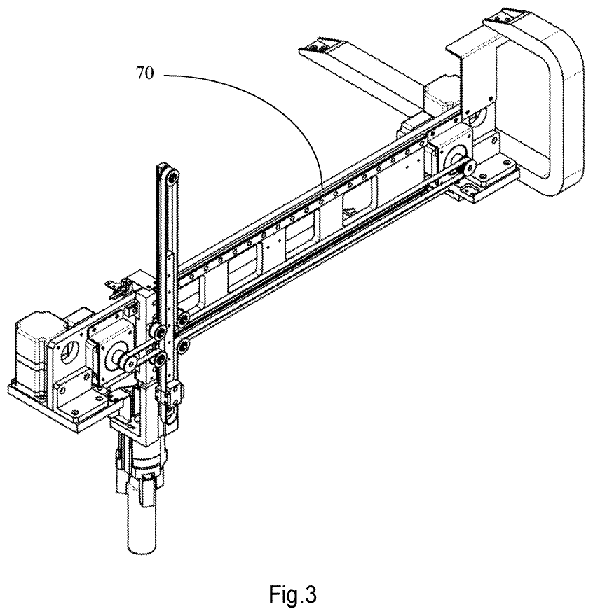

Specifically, the mechanism of the transferring robotic 70 is shown in FIG. 3; the transferring robotic arm 70 is able to move in X-axis, Y-axis and Z-axis directions, respectively, to complete work processing of moving, puncturing and line drawing.

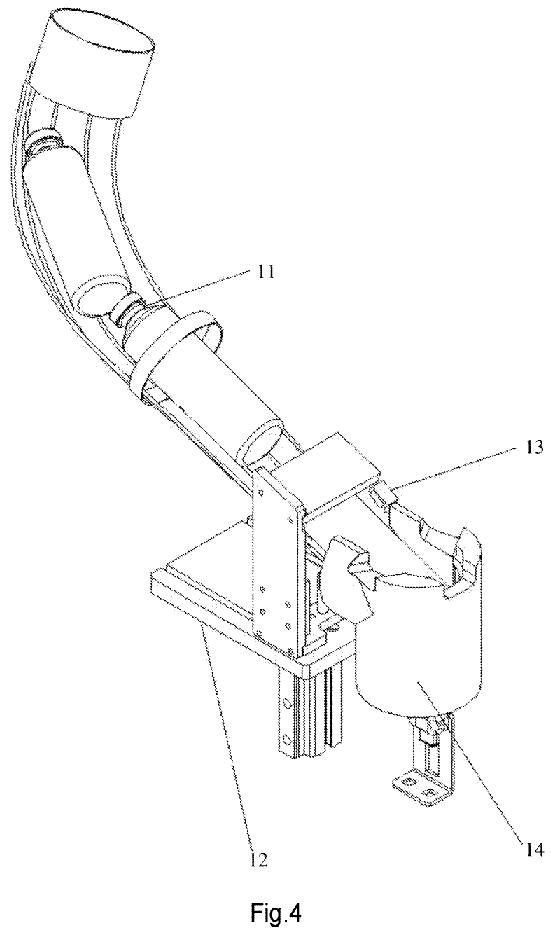

Furthermore, as shown in FIG. 4, the input vertical mechanism 10 comprises a sliding tube 11, a vertical lifting mechanism 12, a detection photoelectric pair 13 and a vertical positioning recessed block 14, the sliding tube 11 being an arc-shaped mechanism, and being used for a positive bottle to slide; the vertical lifting mechanism 12 is arranged on one end of the sliding tube 11, and a piston of a pneumatic cylinder 44 of the vertical lifting mechanism 12 faces the sliding tube 11; the detection photoelectric pair 13 is arranged on the vertical lifting mechanism 12, and is located on one side of the sliding tube 11, and the detection photoelectric pair 13 is electrically connected to a pneumatic cylinder 44 of the vertical lifting mechanism 12; the vertical positioning recessed block 14 is arranged on one side of the vertical lifting mechanism 12 that is remote from the sliding tube 11.

It will be understood that the Merieux microorganism machine ejects a positive bottle, and the positive bottle enters the sliding tube 11 and slides by means of the arc-shaped sliding tube 11 to the vertical lifting mechanism 12; when detecting that a positive bottle has slid to the vertical lifting mechanism 12, the detection photoelectric pair 13 sends a signal to the vertical lifting mechanism 12, and the vertical lifting mechanism 12 lifts up, so that one end of the positive bottle lifts up, and the other end of the positive bottle moves inside the vertical positioning recessed block 14, so that the positive bottle is set vertically in the vertical positioning recessed block 14.

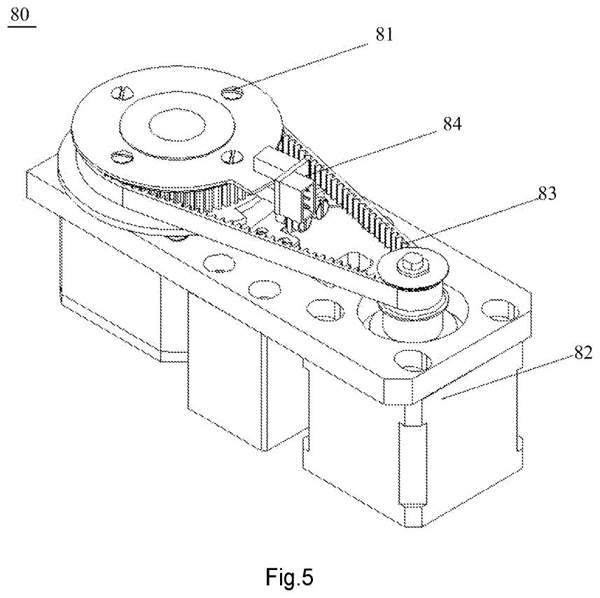

Furthermore, the microorganism positive bottle processing apparatus further comprises two rotation mechanisms 80, the two rotation mechanisms 80 being respectively arranged below the vertical positioning recessed block 14, used for driving rotation of a positive bottle in the vertical positioning recessed block 14, and below the scanning mechanism 30, used for driving rotation of the positive bottle on the scanning mechanism 30; as shown in FIG. 5, the rotation mechanism 80 comprises: a rotating pulley 81, a stepper motor 82 and a rotation photoelectric pair 84, the rotating pulley 81 being arranged below the vertical positioning recessed block 14 and below the scanning mechanism 30, the stepper motor 82 being spaced apart from the rotating pulley 81, and being connected thereto by means of a drive belt 83, and the rotation photoelectric pair 84 being arranged on one side of the rotating pulley 81, and the rotation photoelectric pair 84 being electrically connected to the stepper motor 82.

It will be understood that, by arrangement on the rotation mechanism 80, when the positive bottle inside the vertical positioning recessed block 14 is sterilized, the rotation mechanism 80 (the rotating pulley 81) below the vertical positioning recessed block 14 rotates, thereby driving rotation of the positive bottle, so as to fully sterilize the positive bottle. When it is necessary to scan the positive bottle, the transferring robotic arm 70 moves the positive bottle to the scanning mechanism 30, at which time the rotation mechanism on the lower side of the scanning mechanism 30 rotates, thereby realizing scanning.

Specifically, the rotation photoelectric pair 84 is used for sensing whether a positive bottle has entered the vertical positioning recessed block 14 and the scanning mechanism 30, and transmitting a signal to the stepper motor 82 if a positive bottle is present; the stepper motor 82 rotates, driving rotation of the rotating pulley 81 by means of the drive belt 83, thereby driving rotation of the positive bottle (inside the vertical positioning recessed block 14 and the scanning mechanism 30) on the rotating pulley 81.

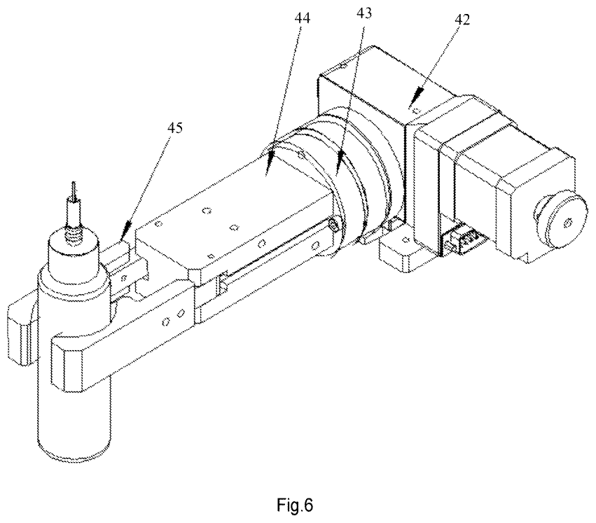

Furthermore, as shown in FIG. 6, the translation and turning mechanism 40 comprises a sliding rail 41, an electric rotating platform 42, a rotating electric motor 43, a pneumatic cylinder 44 and a liquid-dripping rotating gripper 45, the sliding rail 41 being arranged on one side of the scanning mechanism 30, the electric rotating platform 42 being movably arranged on the sliding rail 41, the rotating electric motor 43 being rotatably arranged on the electric rotating platform 42, the pneumatic cylinder 44 being arranged on the rotating electric motor 43, and the liquid-dripping rotating gripper 45 being arranged on a piston of the pneumatic cylinder 44.

It will be understood that the translation and turning mechanism 40 is used for translating a positive bottle and turning a positive bottle; by means of the drive of an electric motor inside the electric rotating platform 42, the electric rotating platform 42 is driven to move on a sliding channel of the sliding rail 41, and, by means of rotation of the rotating electric motor 43, the rotating electric motor 43 and the pneumatic cylinder 44 are driven to rotate relative to the electric rotating platform 42, and the pneumatic cylinder 44 is used for driving the liquid-dripping rotating gripper 45 to close or open.

Furthermore, the microorganism positive bottle processing apparatus further comprises a plate lid-opening mechanism 93 and a plate supply mechanism 94, the plate lid-opening mechanism 93 being arranged on one side of the plate placement mechanism 50, and the plate supply mechanism 94 being spaced apart from the plate placement mechanism 50.

It will be understood that the plate supply mechanism 94 is used for supplying a plate, and the plate lid-opening mechanism 93 is used for opening a lid of a plate; the plate supply mechanism 94 is used for providing and moving a plate.

Furthermore, the microorganism positive bottle processing apparatus further comprises a workbench 91 and a needle supply mechanism 92; the input vertical mechanism 10, the sterilization mechanism 20, the scanning mechanism 30, the translation and turning mechanism 40, the plate placement mechanism 50, the plate lid-opening mechanism 93, the plate supply mechanism 94, the glass slide placement mechanism 60 and the rotating robotic arm 70 are all arranged on the workbench 91; the needle supply mechanism 92 is arranged on the workbench 91.

It will be understood that the workbench 91 may be used for fixing various components; the transferring robotic arm 70 is arranged on an upper part of the workbench 91, located on an upper side of the input vertical mechanism 10, the sterilization mechanism 20, the scanning mechanism 30 and the translation and turning mechanism 40. The needle supply mechanism 92 is used for providing the system with a puncture needle, an inoculating loop and an inoculating needle.

Furthermore, the microorganism positive bottle processing apparatus further comprises: an antimicrobial susceptibility disc dispenser 95, a plate incubation mechanism 96 and a plate loading apparatus 98; the plate loading apparatus 98 is arranged on a lower side of the workbench, used for supplying a plate to the plate supply mechanism 94; the antimicrobial susceptibility disc dispenser 95 is arranged on the workbench; the plate incubation mechanism 96 is spaced apart from the workbench 91, wherein the plate incubation mechanism includes a constant-temperature incubator and a carbon dioxide incubator, the temperature of the constant-temperature incubator and the carbon dioxide incubator both being 35±2° C.

It will be understood that the antimicrobial susceptibility disc dispenser 95 is used for antimicrobial susceptibility testing of a microorganism. Drug susceptibility testing is performed on pathogenic bacteria, and drug selection is determined according to the size of an inhibition ring of an antimicrobial drug to the pathogenic bacteria; an antimicrobial susceptibility disc is placed on an agar surface of an MH Petri dish, completing rapid antimicrobial susceptibility testing.

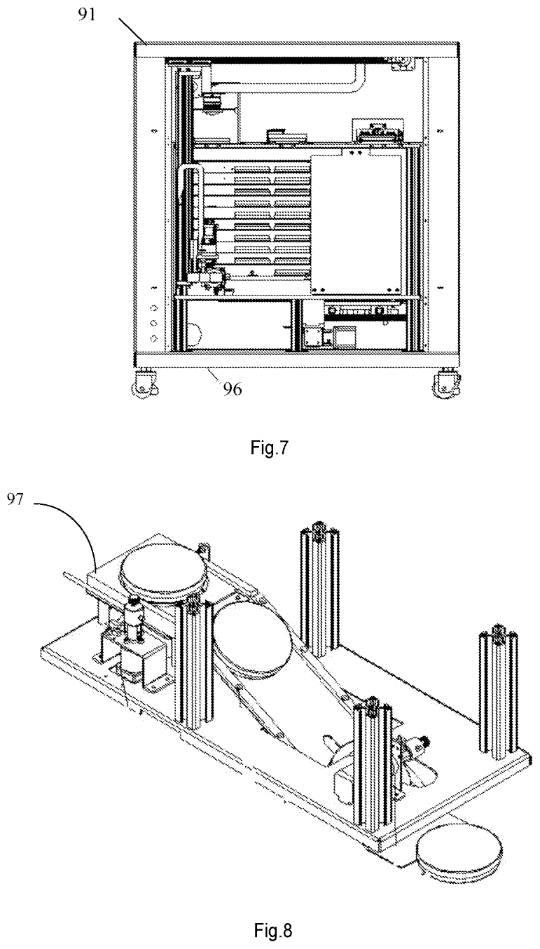

As shown in FIG. 7, the plate incubation mechanism 96 includes a conventional 35±2° C. constant-temperature incubator and a 5% CO₂ incubator, and automatically photographs a plate in a set time, thereby automatically determining the number of antimicrobial susceptibility discs in the MH plate, and transmits the photograph to a client end, facilitating observation of plate bacterial colony growth and antimicrobial susceptibility disc inhibition ring conditions by a laboratory worker. The CO₂ incubator has a sensor that measures CO₂ content, providing constant replenishment and automatic control.

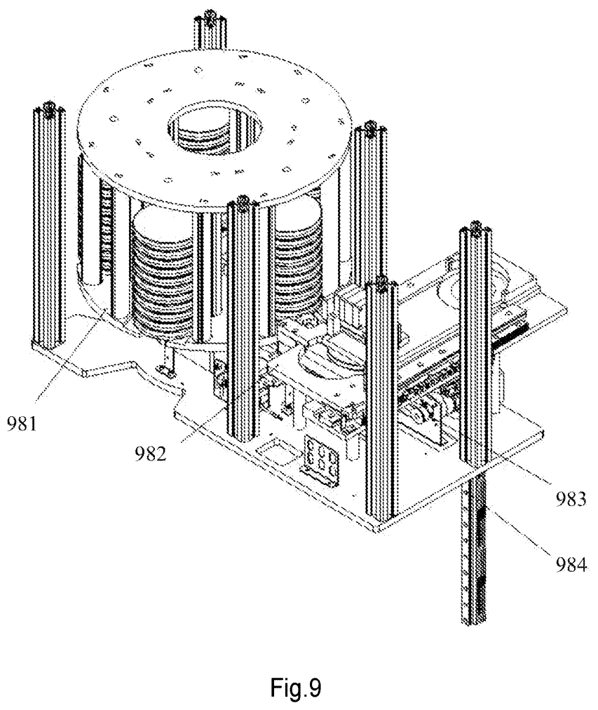

As shown in FIG. 9, a plate loading apparatus 98 comprises a plate storage assembly 981, a plate translation assembly 982, a plate extraction assembly 983 and a raising assembly 984; the plate storage assembly 981 can store various different types of plates, and the plates need to be manually placed. One of the plates in the plate storage assembly 981 is extracted transversely by the plate extraction assembly 983, and then pushed longitudinally by the plate translation assembly 982 to a fixed position of the raising assembly 984, and the raising assembly 984 raises the required plate to the plate supply mechanism 94. Plate loading is then complete.

The plate supply mechanism 94 may output pre-marked plates to the plate incubation mechanism for incubation.

The microorganism positive bottle processing apparatus further comprises a plate output mechanism 97, used for outputting a plate to an incubator; as shown in FIG. 8, the plate output mechanism 97 comprises a rotation output mechanism, a plate slide mechanism and a plate delivery mechanism.

A processing workflow of the microorganism positive bottle processing apparatus: positive bottle input, positive bottle scanning, positive bottle puncturing, positive bottle liquid dripping, drawing a line on a plate, pressing an antimicrobial susceptibility disc into a plate, drawing a line on a glass slide, plate incubation, and plate photography to observe plate bacterial colony growth, the number of antimicrobial susceptibility discs and inhibition ring conditions.

Only preferred particular embodiments of the present invention are described above, but the scope of protection of the present invention is not limited to this; any changes or substitutions that a person skilled in the art could easily conceive of within the technical scope disclosed by the present invention shall all fall within the scope of protection of the present invention. Therefore, the scope of protection of the present invention shall be defined by the scope of protection of the claims.

1. A microorganism positive bottle processing apparatus, comprising:
an input vertical mechanism;
a sterilization mechanism, the sterilization mechanism being spaced apart from the input vertical mechanism, and the sterilization mechanism facing a vertical positioning recessed block of the input vertical mechanism;
a scanning mechanism, the scanning mechanism being spaced apart from the input vertical mechanism;
a translation and turning mechanism, the translation and turning mechanism being arranged on one side of the scanning mechanism that is remote from the input vertical mechanism;
a plate placement mechanism, the plate placement mechanism being arranged on one side of the translation and turning mechanism in a length direction of the translation and turning mechanism;
a glass slide placement mechanism, the glass slide placement mechanism being spaced apart from the plate placement mechanism in the length direction of the translation and turning mechanism; and
a transferring robotic arm, the transferring robotic arm being arranged on one side of the input vertical mechanism, and the transferring robotic arm being used for moving a positive bottle from the input vertical mechanism to the scanning mechanism, moving the positive bottle from the scanning mechanism to the translation and turning mechanism, and for puncturing the positive bottle.

2. The microorganism positive bottle processing apparatus according to claim 1, wherein the input vertical mechanism comprises:
a sliding tube, the sliding tube being an arc-shaped mechanism, used for a positive bottle to slide;
a vertical lifting mechanism, the vertical lifting mechanism being arranged at one end of the sliding tube, and a piston of a pneumatic cylinder of the vertical lifting mechanism facing the sliding tube;
a detection photoelectric pair, the detection photoelectric pair being arranged on the vertical lifting mechanism, and being located on one side of the sliding tube, and the detection photoelectric pair being electrically connected to the pneumatic cylinder of the vertical lifting mechanism; and
a vertical positioning recessed block, the vertical positioning recessed block being arranged on one side of the vertical lifting mechanism that is remote from the sliding tube.

3. The microorganism positive bottle processing apparatus according to claim 2, wherein the microorganism positive bottle processing apparatus further comprises two rotation mechanisms, the two rotation mechanisms being respectively arranged below the vertical positioning recessed block, used for driving rotation of a positive bottle inside the vertical positioning recessed block, and below the scanning mechanism, used for driving rotation of the positive bottle on the scanning mechanism; the rotation mechanism comprises:
a rotating pulley, the rotating pulley being arranged below the vertical positioning recessed block and below the scanning mechanism;
a stepper motor, the stepper motor being spaced apart from the rotating pulley, and being connected thereto by means of a drive belt; and
a rotation photoelectric pair, the rotation photoelectric pair being arranged on one side of the rotating pulley, and the rotation photoelectric pair being electrically connected to the stepper motor.

4. The microorganism positive bottle processing apparatus according to claim 1, wherein the translation and rotation mechanism comprises:
a sliding rail, the sliding rail being arranged on one side of the scanning mechanism;
an electric rotating platform, the electric rotating platform being movably arranged on the sliding rail;
a rotating electric motor, the rotating electric motor being rotatably arranged on the electric rotating platform;
a pneumatic cylinder, the pneumatic cylinder being arranged on the rotating electric motor; and
a liquid-dripping rotating gripper, the liquid-dripping rotating gripper being arranged on a piston of the pneumatic cylinder.

5. The microorganism positive bottle processing apparatus according to claim 1, wherein the microorganism positive bottle processing apparatus further comprises:
a plate lid-opening mechanism, the plate lid-opening mechanism being arranged on one side of the plate placement mechanism; and
a plate supply mechanism, the plate supply mechanism being spaced apart from the plate placement mechanism.

6. The microorganism positive bottle processing apparatus according to claim 1, wherein the microorganism positive bottle processing apparatus further comprises:
a workbench, the input vertical mechanism, the sterilization mechanism, the scanning mechanism, the translation and turning mechanism, the plate placement mechanism, the plate lid-opening mechanism, the plate supply mechanism, the glass slide placement mechanism and the rotating robotic arm all being arranged on the workbench;
a needle supply mechanism, the needle supply mechanism being arranged on the workbench.

7. The microorganism positive bottle processing apparatus according to claim 6, wherein the microorganism positive bottle processing apparatus further comprises:
an antimicrobial susceptibility disc dispenser, the antimicrobial susceptibility disc dispenser being arranged on the workbench;
a plate incubation mechanism, the plate incubation mechanism being spaced apart from the workbench;
a plate loading apparatus, the plate loading apparatus being arranged on a lower side of the workbench, used for supplying a plate to the plate supply mechanism,
wherein the plate incubation mechanism includes a constant-temperature incubator and a carbon dioxide incubator, the temperature of both being 35±2° C.
